(19) United States
(12) Patent Application Publication
(10) Pub. No.: US 2008/0044402 A1
(43) Pub. Date: Feb. 21, 2008

(54) Use of fermented wheat-germ in the feeding and veterinary practice

(76) Inventors: Mate Hidvegi, Budapest (HU); Akos Resetar, Budapest (HU)

Correspondence Address: BIRCH STEWART KOLASCH & BIRCH, PO BOX 747, FALLS CHURCH, VA, 22040-0747, US

(21) Appl. No.: 11/907,794

(22) Filed: Oct. 17, 2007

Related U.S. Application Data
(62) Division of application No. 10/522,148, filed on Jan. 24, 2005.

(51) Int. Cl. A61K 35/00; A61P 29/00

(52) U.S. Cl. 424/115

(57) ABSTRACT

This invention relates to new uses of fermented wheat-germ extract namely for purposes of animal feeding and veterinary therapy. The subject matters of this invention are also the fodders, nutriments, premixes and veterinary preparations containing fermented wheat-germ extract.

This application is a Divisional of co-pending application Ser. No. 10/522,148, filed on Jan. 24, 2005, the entire contents of which is hereby incorporated by reference and for which priority is claimed under 35 U.S.C. §120. Application No. 10/522,148 is the National Phase of PCT/HU02/00082, filed on August 13, 2002, which designated the United States.

The invention relates to new uses of fermented wheat-germ extract, particularly to feeding and veterinary purposes. The subject-matter of the invention includes the fodders, nutriments and premixes containing fermented wheat-germ extract, too.

The fermented wheat-germ extract (hereinafter called VET-HBM) and its production are disclosed in WO 98/08694, where its immunostimulant and metastasis-inhibiting effects are described. (The above-mentioned document is incorporated herein as a reference.) This above mentioned material is produced by fermenting the wheat-germ with Saccharomyces cerevisiae, then by drying the filtered fermentation liquid. The material obtained is characterized by its 2,6-dimethoxy-p-benzoquinone content, which is about 0.4 mg/g dry material.

Surprisingly in the course of our investigations we have found that VET-HBM could be excellently applied in the animal husbandry and animal feeding. VET-HBM provides quicker gain in body weight and increases the power of resistance of animals against diseases, first of all infectious diseases. It serves particularly for increasing the yield of meat and for improving the meat quality of farm animals breeding under circumstances of large-scale production, first of all of poultry and pigs, and at the same time it provides better utilization of the fodder (better feed conversion ratio).

The economical large-scale production of pigs and poultry is an important factor among the factors defining the profitability of the animal husbandry. The reduction of the amount of the fodder has a great importance because of the high fodder prices. In the last decades a lot of good results has been reached in the quantitative and qualitative indicators of the manufactured meat and eggs by the help of the development of professionally compounded fodder and by the help of the application of suitably selected complements. It was proved, however, that the more efficient fodder components, particularly the fodder additives could not applied without anxiety (toxicity, resistance). Namely it was proved, that their effectiveness decreased after certain length of time, and they became objectionable from the environment protecting and, may be, public health point of views. Consequently, the object of this invention is to find body-identical and natural materials which can be applied effectively in feeding and breeding of the farm animals. This object meets the requirement of the European Union to suppress the antibiotics and body-strange materials on the field of yield-enhancers.

In the course of our investigations we tried the above-mentioned natural-base VET-HBM preparation under circumstances of the large-scale production on broiler chickens, geese and turkeys as well as on pigs in the course of the pre-breeding following the weaning and in the period of the fattening. VET-HBM was suitably introduced into the animals' organism by mixing to the usual fodder. In the course of our experiments it was proved that the completion of the starting-, breeding- and finishing nutriments with VET-HBM had favorable effect on the development of broiler chickens, geese, turkeys and pigs because it improved the gain in body weight and the specific fodder utilization, increased the power of resistance and at the same time decreased the environmental pollution. Furthermore, we have found that the vitality of the eggs and consequently the frequency of the hatching increased at the hen stock. Furthermore, we have also found that the aorta cleft which is frequently accompanies with the rapid growth in the turkey fattening, practically could be eliminated; this fact is especially beneficial.

Additionally we have studied the effect of VET-HBM on the infections occurring usually in the poultry farms, and surprisingly it was found that VET-HBM was able to protect the animals against infection caused by Mycoplasma micro-organisms, particularly M. gallisepticum and M. synoviae as well as against coccidiosis caused by Eimeria tenella, and was able to increase the power of resistance against other infections occurring at the poultry (e.g. Gumboro disease).

The contamination of domestic fowl by Mycoplasma gallisepticum and M. synoviae still causes a great economical loss for the poultry husbandry. In the consequence of this infection the gain in body weight and the egg production decrease, the mortality, the hatching loss, the confiscation in the slaughter-house etc. increase. The epithelium injury caused in the respiratory tract supports the secondary bacterial infection, hereby increasing the loss further.

In order to reduce the economical losses there are proposed the use of different antibiotics (e.g. tilozine, thiamuline, norfloxacine, enrofloxacine etc.). In the last years, however, the authorities of different countries have wanted to reduce the use of antibiotics (e.g. the use of some antibiotics, including tilozine, were prohibited as yield-enhancer), and they have insisted to press back the use of such antibiotics which are utilized for human purpose. For this reason we have investigated the effects of VET-HBM, offering very good results in the previous feeding trials, against the wide-spread occurring Mycoplasma infections.

Surprisingly we have found that it was possible to prevent the effect of Mycoplasma infection by dosing VET-HBM similarly as by dosing tiamutin, the best known anti-mycoplasm antibiotics. Because of the considerable incidence of Mycoplasma infection everywhere in the world, this fact has a big economical importance. The economical loss caused by the Mycoplasma infections can be reduced by means of the use of VET-HBM. It can be especially preferred this time when the utilization of the antibiotics is tried to press back in the veterinary practice and in the yield-enhancement.

Additionally, we have made investigations with pigs in connection with M. hyopneumoniae which is present in the usual pig population and causes very great economical loss; surprisingly we have found that VET-HBM was able to protect the pigs against pneumoniae caused by M. hyopneumoniae.

Additionally we have investigated the effect of VET-HBM against another parasitic disease, coccidiosis, caused by Eimeria tenella, which is very wide-spread parasitic disease in the poultry. The number of coccidiosis cases has increased everywhere in the world in the last decades because the mass breeding form has increased the chance of the coccidial infection in the highest degree. It is very likely that 7 Eimeria strains play role in the formation of coccidiosis, among them there are pathogenic and less pathogenic strains. Among the strains causing haemorrhagic enteritis and death, Eimeria tenella, causing appendix coccidiosis, has significant importance. Therefore our investigations have been made on chickens infected with pure Eimeria tenella strain.

Surprisingly we have found that the oocysta defecation of the artificially infected chickens obtained VET-HBM complement significantly decreased as compared with the controls; this means that the intermediate forms destroyed, and the E. tenella parasites causing great damage in the appendix were not able to develop. Consequently VET-HBM can be able to protect the chickens against the serious disease caused by E. tenella.

In addition to foregoings we have studied the effect of VET-HBM on the change of the antibody-level of chickens vaccinated against Gumboro-disease (by CEVAC-vaccine). We have found that VET-HBM dosed to the fodder increased significantly the antibody production of chickens, and together with the increase of the antibody level it enhanced the effect of CEVAC vaccine in the period of the chicken breeding providing protection of higher degree to the animals.

Further we have observed that the dosing of VET-HBM reduced significantly the economical loss caused by stress effects (e.g. heat- and transport stress).

On the basis of the above-mentioned findings the invention relates to the use of fermented wheat-germ extract as fodder complement for the production of fodders, nutriments or premixes for animals. In the terms used in this invention the term “animal” means first of all farm animals, as cattles, horses, pigs, poultry, rabbits, cultivated fishes; pets, as dogs, cats and other domestic animals; as well as zoo animals. The fodder complement according to this invention can be used as yield-enhancer for farm animals, preferably poultry, as broiler chicken, hen, roasting goose, feather goose, liver goose, roasting duck, turkey, as well as pigs and piglets.

According to another aspect the invention relates to fodders, nutriments and premixes, which contain fermented wheat-germ extract in addition to known fodder-, nutriment- and premix components. The fodders and nutriments of this invention contain the wheat-germ extract in an amount of about 0.001-10% by weight, preferably 0.01-5.0% by weight, and most preferably 0.3-1.0% by weight. The fodder and nutriment premixes according to the invention can contain wheat-germ extract in an amount of about 0.001-50% by weight. The preparation according to the invention will be prepared in that manner that the wheat-germ extract fermented in a manner known per se is mixed in the abovementioned amount with solid, forgeable vehicles, in the case of premixes with usual vitamins and micro-elements and fodder, respectively.

The VET-HBM complement according to this invention is applied mixing with the starting, breeding and finishing fodder or nutriment, respectively, partially or in the course of whole breeding. VET-HBM can also be applied dosing to the drinking water of the animals. According to another aspect the invention relates to a method for yield-enhancing of farm animals. According to this method fermented wheat-germ extract as yield-enhancer is given to the fodder of the animals and the animals are fed with this fodder. The above-mentioned yield-enhancer is applied in an amount of 0.1-6 g/fodder kg, preferably 0.3-3 g/fodder kg.

According to another aspect the invention relates to the use of fermented wheat-germ extract in animals for preventing and/or decreasing of Mycoplasma infection, infectious inflammations and coccidiosis infections of poultry, and for increasing the antibody titer of vaccinated poultry. The fermented wheat-germ extract can be employed advantageously to prevent Mycoplasma gallisepticum or Mycoplasma synoviae infections, to prevent and/or decrease the coccidiosis infection of poultry, as well as to prevent the pneumoniae caused by M. hyopneumoniae at pigs. The invention relates to the use of the fermented wheat-germ extract in manufacturing preparations for the abovementioned purposes.

The veterinary preparations according to the invention containing fermented wheat-germ extract can be prepared in usual manner, in the course of which the active ingredient mixing with one or more veterinary acceptable auxiliary materials will be formed to preparations enhancing power of resistance of animals, to preparations preventing and/or treating Mycoplasma infections and infectious inflammations, to preparations preventing and/or treating poultry coccidiosis infections and to preparations increasing antibody titer value of vaccinated poultry.

The preparations using auxiliary materials usually applied in the veterinary practice can be formulated to tablets, pills, capsules, gels or pastes. These auxiliary materials include gelatin, natural sugars as row sugar, lactose, maltose and dextrose, lecithin, pectin, cyclodextrin, dextran, polyvinylpyrrolidone, polyvinyl acetate, acacia gum, xanthane gum, tragacanth, agar-agar, alginic acid, carboxymethyl cellulose, carboxymethyl cellulose sodium, hydroxypropyl cellulose, hydroxypropyl methyl cellulose or similar cellulose derivatives, emulsifiers, oils, fats, particularly glycerol esters and polyglycerol esters derived from saturated fatty acids.

The amount of the components in the preparation can be varied and it depends on various factors as on individual demands of the animals to be treated. The doses to be administered can depend, inter alia, on the size of the animal to be treated and the type of the disease to be prevented or treated. The daily dose can be administered in a single dose or dividing to more part doses in a day.

The invention is further illustrated in the following examples, which, however, are not construed as limiting.

### EXAMPLES

VET-HBM employed in the following examples was prepared according to the following technology which substantially corresponds to Example 2 of WO 99/08694.

300 kg wheat germ ground to flour quality (according to the Hungarian standard) and 100 kg yeast (Saccaromyces cerevisiae) were placed in a 5 m³ fermentor, and drinking water was added until the volume became 4000 I. The fermentation period was 18 hours, during which continuous aerating (0.5 I air/I fermented liquid/minute) and slow stirring (30 rev./min) was used. In order to inhibit foaming 1 I/m³ sunflower oil was added to the mixture. After fermentation aerating and stirring were discontinued, and the fermented liquid was separated first in a screw decanter, then in separator and finally in a sharpening separator.

Preparation of Fraction 1.

The fermented liquid was filtered sharp and the sharpness was checked by microscope. The filtered fermented liquid contained practically no cells, which meant that maximum 1 yeast cell was found per 10 sights. The resulting fermented liquid, which contained about 1.5% by weight dry material was evaporated in a vacuum condenser at a temperature of 40-50° C. and after discontinuing the vacuum was boiled at atmospheric pressure for about 15 minutes. After this the dry material content of the solution was determined and so much maltodextrin—first dissolved in hot water and then cooled—was added that the dry matter content of a solution became about 30% by weight. After this the solution was spray dried in a shear nozzle rotating spay drier in which the temperature of the outgoing air was about 90° C. The resulting final product as a powder contained 60% by weight of the fermented vegetal material according to the invention and 40% by weight maltodextrin. The dimethoxy-p-benzoquinone content which was determined by HPLC, was 0.15 mg/g dry material ±20%.

Preparation of Fraction 2.

The biomass with 25-27% by weight dry material separated on the screw de-canter was dried in the ratio of 1:1 on a finely ground flaked maize carrier in a fluidization drier equipment, and its grain size was adjusted between 0.2-0.8 mm by granulation.

Preparation of Final Product

The fraction 1. and fraction 2. were combined in a homogenizer of Lödige system and carefully homogenized. The 2,6-dimethoxy-p-benzoquinone content of the preparation obtained in this manner was 0.11 mg/g dry material ±20%.

### Example 1

32600 1 day old broiler chicks (Shaver Starbo) were drawn into the feeding experiment from them three groups were formed. The control group “K” consisted of 16300, the two experimental groups (“I” and “II”) consisted of 8150-8150 baby chicks. The inner content of the fodder corresponded to the necessary values prescribed to the current breeding stage. The VET-HBM standardized preparation was mixed to the starting-, breeding- and finishing nutriment of the animals of the experimental groups “I” and “II” in an amount of 3 g/fodder kg.

The animals of control “K” group and experimental “II” group obtained enrofloxacine [Avian Pathol. 19, 511-522 (1990)] together with the drinking water in the 3-5. days to prevent the bacterial infections. The animals of experimental group “I” did not obtained enrofloxacine and any other medicinal treatment otherwise usual in the chick breeding. To prevent the Gumboro-disease CEVAC vaccine (Phylaxia, Budapest, Hungary) was mixed to the drinking water of the animals equally in all the three groups.

The feeder system was governed automatically and was connected to two tanks able to receive 10-10 tons of fodder. The change of fodder occurred gradually. The litter was about 120 m³ dry pine chip spreaded in 6 cm depth which corresponds about 100 tons of manure at every change of litter. The ventilation was solved by 44 ventilators supplied with speed governor; the capacity of ventilators was individually 10000 m³/hour.

Results

1. Evaluation of Deaths Numerically and in Percentage:

In the broiler chicken feeding experiment the hatching weakness as well as the unusually hot summer temperature (heat shock) caused a bigger death than usual (5.3%). The death in the course of the experiment was in the control group 5.57% (913), in the experimental group “I” 4.9% (400), and in the experimental group “II” 4.04% (330).

Therefore it was observable on the basis of the deaths that in the prevention of the heat shock VET-HBM gave a significant help for the animals.

2. Evaluation of Weekly Gain in Body Weight in Percentage:

In the course of the feeding experiment the gain in body weight breaking down to breeding weeks was bigger in every case at the experimental animals obtained VET-HBM auxiliary material than that of controls. The results of the weekly mass measuring and the weekly gain in weight in the percentage of the control are shown in Table 1 below.

TABLE 1
The result of chicken's weekly body weight measuring

| Breeding | Group | Group | Gain in | Group | Gain in |
|---|---|---|---|---|---|
| week | “K” (g) | “I” (g) | weight (%) | “II” (g) | weight (%) |
| 1. | 150 | 153 | 102.0 | 157 | 104.66 |
| 2. | 372 | 375 | 100.8 | 379 | 101.88 |
| 3. | 736 | 740 | 100.5 | 749 | 101.76 |
| 4. | 1232 | 1276 | 103.57 | 1288 | 104.54 |
| 5. | 1580 | 1589 | 100.56 | 1597 | 101.07 |
| 6. | 1953 | 2001 | 102.45 | 2055 | 105.22 |

3. Evaluation of the Formation of Fattening Indices:

It can be seen from Table 2. below that the gain in body weight of the animals obtained VET-HBM auxiliary material exceeded the values of the control group at the end of the experiment, with 2.45% in group “I” and with 5.22% in group “II”.

Conversely, the specific utilization (feed conversion ratio) of fodder was of less value in the experimental groups that in the case of control animals in group “K” , not obtained auxiliary material, with almost 12% (1.85 kg/kg) in the experimental group “I” , and with 12.4% (1.84 kg/kg) in the experimental group “II”.

TABLE 2
The effects of VET-HBM in the course of
large-scale breeding of broiler chickens

| | Control group | Experimental | Experimental |
|---|---|---|---|
| Measurings | “K” | group “I” | group “II” |
| Initial number of animals | n = 16300 | n = 8150 | n = 8150 |
| (100%) | | | |
| Initial average body weight | 0.055 | 0.053 | 0.052 |
| (kg) | total: 896.5 | total: 431.9 | total: 423.8 |
| Final number of animals in the | n = 15370 | a = 7750 | n = 7820 |
| percentage of initial number | 94.00% | 95.09% | 95.95% |
| Final total body weight | 30017.6 | 15507.7 | 16070.1 |
| (kg) | | | |
| Final average body weight (kg) | 1.953 | 2.001 | 2.055 |
| in the percentage of control | 100% | 102.45% | 105.22% |
| Total fodder spent | 61154.3 | 27890.1 | 28789.2 |
| (kg) | | | |
| Feed conversion ratio (kg/kg) in | 2.10 | 1.85 | 1.84 |
| the percentage of control | 100% | 88.1% (−11.9) | 87.6% (−12.4) |

In addition the dead-line for delivery of the broiler chickens shortened with 1 week and the slaughtering experiments confirmed that the yield of the lean meat, particularly the mass of breast meat and leg meat, increased.

It should be underlined that the medical treatment applied till now in the usual breeding technology was withdrawn from the experimental group “I” and the animals obtained only VET-HBM. Despite of this fact these chickens proved to be such resistant as the members of the experimental group “II” which obtained the usual breeding technology complemented with VET-HBM.

The consistence of the feces changed, the number of diarrhea cases de-creased and the consistence of the litter improved because of the defecation of harder feces. It has a great role from the point of view of the environment protection, because it is necessary to change the litter of great quantity rather more rarely, which fact results in savings in material and manpower.

We have got similar results when VET-HBM was applied in an amount of 0.3 g/fodder kg.

### Example 2

In a pig court, under the circumstances of the large-scale production, three groups of 35 days old weaned bacon piglets were down into the experiment, 50-50 in all the groups. In the course of an almost 60 days feeding pre-experiment 3 g VET-HBM were mixed to 1 kilogram fodder of the animals. The animals of the control group were fed with a fodder which was usual till now in this manufacture. The two groups of the experimental animals, however, consumed a fodder containing VET-HBM auxiliary material, from the age of 35 days tilt the age of 92 days.

At the weaning the litters of the animals were divided into two groups among them the first formed the control group “A” and the second formed the groups “B” and “C” of the experimental animals, hereby the genetic factors were eliminated. In this experiment all the three groups of the animals were of mixed sex. In the course of the raising that breeding-, feeding- and drinking technology was employed which was usual in this manufacture. The piglets obtained Starter piglet nutriment from the age of 35 days till the age of 95 days. The dosage of the fodder was performed by a feeder equipment of Big Dutchman MC44-V03 system. The daily fodder consumption was registered by a feeding computer of LCD SCAN type. In the course of the experiment the followings were registered in both the control and experimental groups:
initial and final number of the animals,
body weight of the animals at the beginning of the experiment,
fodder consumption group by group,
changes occurring in the sanitary, clinical state of the animals, and the reasons of the occasional diseases and deaths,
closing average individual body weight and total weight (weighing individually, living mass).

The result are shown in Table 3.

TABLE 3
The effect of VET-HBM to the breeding of piglets

| | Control group | Experimental | Experimental |
|---|---|---|---|
| Measurings | “A” | group “B” | group “C” |
| Initiate number of animals | n = 50 | n = 50 | n = 50 |
| (100%) | | | |
| Initial average body weight | 12.26 | 12.04 | 12.05 |
| (kg) | total: 613 | total: 602 | total: 602.5 |
| Final number of animals in the | n = 47 | a = 49 | n = 50 |
| Percentage of initial number | 94% | 98% | 100% |
| Final total body weight | 1385.5 | 1523.9 | 1613.0 |
| (kg) | | | |
| Final average body weight (kg) | 29.48 | 31.10 | 32.26 |
| in the percentage of control | 100% | 105.4% | 109.43% |
| Total fodder spent | 1637.7 | 1843.8 | 1930.0 |
| (kg) | | | |
| Feed conversion ratio (kg/kg) in | 2.12 | 2.00 | 1.91 |
| the percentage of control | 100% | 94.33% (−5.67) | 90.09% (−9.91) |

It can be seen from Table 3, that the completion of the fodder of the piglets with VET-HBM reduced the level of deaths. Between of ages of 35 days and 92 days of the piglets the VET-HBM charged with the fodder affected on the gain in weight of the animals, namely the body weight of the piglets of group “B” exceeded that of the control animals with 5.4% (31.10 kg), while the body weight of the piglets of group “C” exceeded that of the control animals with 9.43% (32.26 kg).

The feed conversion ratio (specific utilization of the fodder) was lower than at the control animals failed to get complement (“A”), namely with 5.67% at the animals of group “B” and with 9.91 % at the animals of group “C”.

The consistence of the feces changed, diarrhea did not occur at all at the piglets of the experimental group. The consistence of the litter was all the time better than at the animals of the control group because of the hard feces defecation.

### Example 3

On the basis of the favorable results the dosage of VET-HBM was continued until finishing of the fattening. From the 95. day the pigs consumed fattening nutriment in the course of fattening until the day of the slaughtering (that is during 172 days). The fodder contained 3 g VET-HBM in this case, too. The result are shown in Table 4.

As it can be seen from Table 4, there was no death at all until finishing the fattening. The finishing body weight of the pigs was higher than that of the control (108 kg), namely with 1.8% (110 kg) in group “B” and with 5.5% (114 kg) in group “C”.

The feed conversion ratio (specific utilization of the fodder) was lower at animals of the experimental group than at the control animals, namely with 10.9 and 15.2%, respectively. The consistence of the feces changed, diarrhea did not occur at the experimental animals. In the case of the control animals, however, there occurred diarrhea pigs.

TABLE 4
The effect of VET-HBM on the breeding of fattening pigs

| | Control group | Experimental | Experimental |
|---|---|---|---|
| Measurings | “A” | group “B” | group “C” |
| Initial number of animals | n = 47 | n = 49 | n = 50 |
| (100%) | | | |
| Initial average body weight | 29.48 | 31.10 | 32.26 |
| (kg) | total: 1385.56 | total: 1523.9 | total: 1613 |
| Final number of animals in the | n = 47 | a = 49 | n = 50 |
| percentage of initial number | 100% | 100% | 100% |
| Final total body weight | 5076 | 5390 | 5700 |
| (kg) | | | |
| Final average body weight (kg) | 108 | 110 | 114 |
| in the percentage of control | 100% | 101.8% (+1.8) | 105.5% (+5.5) |
| Total fodder spent | 12213.9 | 11404.7 | 11484.4 |
| (kg) | | | |
| Feed conversion ratio (kg/kg) in | 3.31 | 2.95 | 2.81 |
| the percentage of control | 100% | 89.1% (−10.9) | 84.8% (−15.2) |

### Example 4

Feeding experiments were performed under circumstances of large-scale pro-duction on roasting geese, and the effects of VET-HBM were studied. 250-250 first-class, freshly hatched baby geese of mixed sex were drawn into the experiment, where one of the groups provided the experimental group and another provided the control group. The inner content parameters of the fodders corresponded to the necessary values prescribed to the current breeding stages. The VET-HBM auxiliary material in the experimental group was mixed to the starting-, breeding- and finishing nutriment of the animals in an amount of 0.3 g/fodder kg.

The housing of the animals corresponded to the current prescriptions of the goose breeding (number of birds: 8 birds/m²). The 32° C. room temperature was reduced gradually to 20-22° C. from the 3. day following the admission until the 14. day. The natural lighting in the course of the pre-breeding period was complemented with artificial lighting. The intake of water of the animals in the course of the pre-breeding period (4 weeks) was performed through a tipped drinking device then from a piped drinking device and libitum.

In course of the experimental period the following parameters were registered in both groups:
initial and final number of the animals,
clinical state,
death loss, indicating the reasons of the deaths, too,
individual body weight in the ages of 28th and 55th days, and
feed conversion ratio in the 28th and 55th days.

The results are shown in Table 5.

From the results obtained it can be seen that in the course of goose breeding the nutriments complemented with fermented wheat-germ extract can be applied very effectively.

TABLE 5
The effect of VET-HBM in the course of the roasting goose breeding

| Measurings | Control group | Experimental group | |
|---|---|---|---|
| Initial number of animals | n = 250 | n = 250 | |
| (100%) | | | |
| Initial average body weight | 0.087 | 0.087 | |
| (kg) | total: 21.75 | total: 21.75 | |
| Final number of animals in the percent- | n = 238 | n = 241 | |
| age of control | 95.2% | 96.4% | (+1.2%) |
| Final total body weight | 1193.33 | 1265.00 | |
| (kg) | | | |
| Average body weight on the 28th day | 2.030 | 2.167 | |
| (kg) in the percentage of control | 100% | 106.74 | (+6.74) |
| Average body weight on the 55th day | 5.014 | 5.249 | |
| (kg) in the percentage of control | 100% | 104.68% | (+4.68) |
| Feed conversion ratio (kg/kg) on the 28th | 2.52 | 1.59 | |
| day in the percentage of control | 100% | 94.09% | (−5.91) |
| Feed conversion ratio (kg/kg) on the 55th | 2.78 | 2.52 | |
| day in the percentage of control | 100% | 90.65% | (−9.35%) |

The clinical state of the experimental animals did not show any deviation compared to the control animals. The deaths in the experimental group in the pre-breeding period lasting 8 weeks decreased to 3.6% compared to the 4.8% value of the control group. The experimental group exhibited significant gain in the body weight as compared to the control group. Until the 28th day the gain in the body weight of the experimental group was better with 6.7% than that of the control group, while on the 55th day of life the body weight of the roasting geese in the experimental group exceeded that of the animals of the control group with 4.7%.

The utilization of fodder of the experimental group improved significantly. The feed conversion ratio (specific fodder utilization) was better in the experimental group than in the control group, namely with 5.9% in the first 28 days and with 9,35% until the 55th day of life.

### Example 5

Feeding experiment was performed on broiler turkeys under circum-stances of large-scale production and the effect of the feeding of the fodder complemented with VET-HBM was investigated.

The experiment was performed with 1 day old baby turkey chicks divided into 4 groups. In the control group 9300 hen (A) and 8700 cock (C) turkey chicks included while into the experimental groups 9600 hen (B) and 9100 cock (D) turkey chicks of meat type (BIG-6) arrived. To the fodder of the animals of group B and D 0.3 g VET-HBM was given per kg of fodder.

The experiment was performed in a turkey farm of large scale where 1 day old meat hybrid turkey chicks designated BIG-6 (Gigant) (the place of origin: Nadudvar, Hungary) were introduced into the abovementioned groups. The settling density was the same at all the four groups (4 animals/m²). In the buildings with deep littering breeding technology the temperature of room, the ventilation and the moisture were guaranteed corresponding the current ages, according to the technological prescriptions. The fodders of the broiler turkeys were the starting-, breeding- and finishing turkey nutriments usual in the turkey breeding and fattening, respectively [and they were assembled according to the prescriptions of the Hungarian Fodder Code (1990)]; these fodders were complemented with 0.3 g VET-HBM per kilogram in the experimental groups B and D.

The animals obtained the starting nutriment until 56th day of their life, the breeding nutriment from 57th until 112nd day of their life and the finishing nutriment from 113rd day of their life until the end of the fattening. For the prevention and treating of the bacterial infection Lincospectin usual in the turkey fattening was employed in all the four groups.

In the course of the experiment the followings were registered both in control and experimental groups:
initial and final number of the animals,
death loss, indicating the reasons of the deaths, too,
body weight at the start and at the end of the experiment,
changes occurring in the health, clinical state,
technological faults in the course of the breeding, and
data of the fodder utilization.

The results obtained are shown in Table 6.

TABLE 6
The effects of VET-HBM in the course of the turkey fattening

| | Hen | | | Cock | | |
|---|---|---|---|---|---|---|
| | Control | Experimental | | 1 Control | Experimental | |
| Measurings | (A) | (B) | | (C) | (D) | |
| Initial number of animals | n = 9300 | n = 9600 | | n = 8700 | n = 9100 | |
| Initial total body weight | 586 | 595 | | 539 | 564 | |
| (kg) | | | | | | |
| Initial average body | 0.063 | 0.062 | | 0.062 | 0.062 | |
| weight (kg) | | | | | | |
| Final number of animals | n = 8804 | n = 9307 | | n = 8192 | n = 8714 | |
| in the percentage of control | 94.66% | 96.94% | (+2.28%) | 94.16% | 95.75% | (+1.5$%) |
| Final total body weight | 77823 | 91581 | | 138772 | 161906 | |
| Final average body | 8.76 | 9.84 | | 16.94 | 18.58 | |
| weight in the percentage | 100% | 112.32% | | 100% | 109.68% | |
| of control | | | | | | |
| Feed conversion ratio (kg | 3.27 | 3.07 | | 3.24 | 3.05 | |
| feed/kg weight gain) in | 100% | 93.88% | (−6.12) | 100% | 94.13 | (−5.87) |
| the percentage of control | | | | | | |

The clinical state of the experimental animals did not show any deviation compared to the control animals. More animals died among the animals of the control group with 2.28% and 1.58%, respectively.

At the end of the experiment the gain in body weight was bigger in the two experimental groups, namely with 12.32% in group B and with 9.68% in group C, respectively.

The feed conversion ratio was better in the two experimental groups, too; it was less with 6.12% in group B and with 5.87% in group D, respectively, compared to the control groups.

It should be underlined as a great advantage the fact that in the course of the turkey fattening the aorta clefts and deaths because of them, frequently occurring due to rapid growth, practically ceased to exist in the consequence of the dosage of VET-HBM.

### Example 6

### Investigation of the Effect Against Mycoplasma gallisepticum

The investigations were performed with Arbor Acress chickens of meat type free of M. synoviae infection. The freedom from Mycoplasma synoviae of the animals was verified with systematic serological screening examination in an agglutination test by the help of M. gallisepticum and M. synoviae antigens (Intervet International B.V.M.; Boxmeer, The Netherlands). Furthermore, the animals were tested in an ELISA-test based on monoclonal antibodies and with the use of MYGA test-kit (Diagnosztikum Kft; Budapest, Hungary) and MYSA kit (Svanova, Uppsala, Sweden) [Czifra, Gy. et al.: Avian Dis. 37, 680-688 (1993)]. The serological examinations gave negative results. Furthermore, from the same hatching from which the experimental animals derived, isolation of Mycoplasma was tried from the nasal cavity, trachea and air pocket of 20 1 day old chicks using medium B [Erno H., and Stipkovits, L.: Acta Vet. Scand. 14, 436-449 (1973)] and Frey's media [Frey, M. C. et al.: Am. J. Vet. Res. 29, 2164-2171 (1968)]. This cultivation closed with a negative result, too.

120 animals were drawn into the experiment divided into 4 equal groups with same number (30-30 animals) in that manner that the average body weight of the 4 groups did not deviate from Bach other in a Student t test.

For the infection of the experimental animals M. gallisepticum N^{o} 1226 was employed which had been previously amplified in medium B for 24 hours. The germ content was 9.5×10⁸ pfu/ml (pfu=plaque forming unit).

The experimental groups were treated and infected as follows:

Group 1. was located in a 200 litre box which can be fastened down hermetically, into which 10 ml sterile medium B was sprayed, then the animals were kept in this box for 20 minutes. Hereafter this group was located in a separate room and the animals did not get any treatment; this group was considered as a negative control.

Group 2. was located in an identical box into which 10 ml M. gallisepticum broth culture was sprayed, then the animals were kept in this box for 20 minutes, Hereafter this group was located in a separate room and the animals did not get any treatment; this group was considered as a control for the monitoring of the infection.

Group 3. was infected in identical manner as Group 2., then after location in a third room the animals were fed with a chick breeding nutriment containing VET-HBM in 3 g/kg concentration in the course of the experiment.

Group 4. was infected in identical manner as Group 2., then after location in a fourth room the animals were fed with a nutriment containing 200 mg/kg tiamutin (Biochemie GmbH, Kundl, Austria) in the course of the experiment.

To the judgment of the effectiveness of the treatment the following parameters were investigated: clinical symptoms, changes in body weight, feed conversion ratio; in addition to them pathological, histological and serological examinations as well as Mycoplasma re-isolation were performed. In the course of their evaluation the following result were obtained.

Results

1. Clinical Examination

The clinical symptoms and the possibly deaths were investigated each day. There were no any clinical symptoms at the treated animals (Group 3. and 4.) while respiratory symptoms appeared in the infected, non-treated group from the 6. day, moreover, 1-1 death occurred in the 7. and 9. day, too.

2. Gain in Body Weight

The gain in body weight was statistically significantly lower in the infected, non-treated group than in the control, non-treated group as well as in the two treated groups. At the same time in the two treated groups the gain was of same degree as in the control group.

3. Feed Conversion Ratio

The feed conversion ratio increased in the infected, non-treated group with 0.45 kg/kg while it remained at the same level in both treated groups as in the control group.

4. Pathological Examination

At the end of the experiment all the animals were examined by pathological dissection to air-pocket- and peritoneum inflammation characteristic of the M. gallisepticum infection.

In the Group 1. all the animals were negative while in the Group 2. all the animals exhibited air-pocket- and peritoneum inflammation of different severity. In the treated Group 3. and Group 4. pathological alterations developed significantly more scarcely and their severity was considerably more moderate than in the animals of Group 2. The results of the groups treated with VET-HBM and tiamutin, respectively, did not differ from each other.

5. Histological Examinations

In the Group 2. in consequence of the infection the number of lymphohystiocytic bronchitis and lobular interstitial pneumoniae increased significantly as compared to the non-infected Group 1. At the same time the parameters in the Group 3. and Group 4. treated VET-HBM and tiamutin, respectively, remained on the same level as in the Group 1., with the exception of the lobular interstitial pneumoniae the number of which was significantly higher in the Group 3., than in the control group. The Group 3. and Group 4. did not differ from each other statistically as far as the alterations examined concerned.

6. Serological Examination

The blood plasma of all the chickens was investigated on slide in a M. gallisepticum agglutination test. The severity of the reaction was scored, and the number of respondent animals and the sum of the scores were compared in a Chie square test. The Group 1. remained negative till the end of the experiment. In the treated Group 3. and Group 4. significantly less animal exhibited serological response (6 and 8, respectively, against 25), and the scores were significantly lower (6 and 11 respectively) than in the non-treated Group 2. (75 scores).

7. Mycoplasma Re-Isolation

The re-isolation of the infectious Mycoplasma strain was performed as follows. Following of the infection after 1 hour 5-5 animals were killed. 1-1 cm long pieces of trachea were placed into 2 ml liquid medium B, and after 3 minutes of shaking germ counting were performed in the medium. At the end of the experiment re-isolation of the strain used for infection was attempted from the respiratory organs (trachea, lung, air-pocket) and other organs (brain, liver, spleen, kidney, heart) of all the chickens in that manner that samples were carried from every abovementioned organ to solid medium B by the help of a tampon. The agar slants were cultivated for 10 days then they were evaluated. A portion of the isolates was identified with an epifluorescent method using a specific immune serum.

Immediately after the infection Mycoplasma was not successfully isolated from the trachea of the animals of the Group 1. In the same time it was possible to show 1×102-2.7×10³ pfu/ml M. gallisepticum from the trachea of the infected animals of the Group 2.

At the end of the experiment it was not possible to recultivate the strains employed to the infection from the Group 1., while from the Group 2. it was possible to recultivate them in 64 instances first of all from the trachea, lung and air-pockets. On the other hand the re-isolation was successful significantly more scarcely from the Group 3. and Group 4. (by 10 and 3 occasions, respectively), and only from some lungs and tracheae but not from other inner organs. Substantial difference could not be observed between the groups treated VET-HBM and tiamutin, respectively.

### Example 7

### Examination of the Effect Against E. tenella

48 1 day old chickens were drawn into the experiment divided into four groups (12-12 animals in every group). The chicks were housed group by group in cages; the temperature of the room was 28° C. in the course of the experiment. In the course of the breeding the animals consumed usual starting nutriment and drinking water ad libitum until their age of 14 days (Group 1. and Group 2.). The nutriment of Group 3. and Group 4. was complemented with 0.3 g VET-HBM per kilogram fodder.

The animals of the Group 2. and Group 4. were infected per os by a suspension containing 2×10³ Eimeria tenella sporulated oocysts.

Starting at the 7. day from the infection the oocyst defecation was investigated in the faeces. The daily amount of faeces of the animals belonging to the same group was weighed and homogenized individually. The same amount was weighed out from Bach animal's faeces and it was homogenized with 2.5% K₂Cr₂O₇ solution. The daily oocyst defecation of a given group was determined in McMaster chamber with three times repetition. The result are given in Table 7.

TABLE 7
Determination of daily oocyst defecation in McMaster chamber

| Control group | | Treated group | |
|---|---|---|---|
| | Group 2. | | Group 4. |
| Group 1. | Infected, average ± | Group 3. | Infected, average ± |
| Non-infected | SD | Non-infected | SD |
| 1. day 0 | 21500 ± 0.02 | 1. day 0 | 23 800 ± 0.03 |
| 2. day 0 | 120 500 ± 0.31 | 2. day 0 | 27 250 ± 0.07 |
| | | | p < 0.0001 |
| 3. day 0 | 84 500 ± 0.11 | 3. day 0 | 20 300 ± 0.08 |
| | | | p < 0.0001 |
| 4. day 0 | 75 800 ± 0.22 | 4. day 0 | 15 900 ± 0.14 |
| | | | p < 0.0001 |
| 5. day 0 | 5 700 ± 0.13 | 5. day 0 | 1 800 ± 0.02 |
| | | | p < 0.0001 |
| 6. day 0 | 950 ± 0.03 | 6. day 0 | 200 ± 0.01 |
| | | | p < 0.0001 |
| 7. day 0 | 350 ± 0.02 | 7. day 0 | 15 ± 0.01 |
| | | | p < 0.0001 |

It can be seen from the foregoing Table 7 that the development of the oocysts in the infected group 4 obtained VET-HBM was significantly less (p<0.0001 and p<0.001) than at the animals of the infected group 2 living on traditional nutriment. In this group the rise of the oocyst defecation considerably exceeded the treated group for days and this higher level remained till the end of the experiment.

The body weight measurings in the 0., 7th and 14th days verified that the body weight at the artificially infected animals living on traditional nutriment decreased gradually until the finishing day of the experiment while in the groups of the infected and non-infected chicks consuming VET-HBM significant (p<0.001) gain was registered in the body weight.

### Example 8

### Measuring of the Antibody Level of Vaccinated Chickens

Group by group 7-7 1 day old chicks of Ross-308 type (source of supply: Babolna, Hungary) were drawn into the experiment which chicks once had been treated in the eggs with a vaccine against infectious Gumboro-disease (CEVAC vaccine from Phylaxia, Budapest, Hungary). To the basic nutriment of the half of the chicken stock VET-HBM was given in an amount of 0.3 g/fodder kg. The animals consumed ad libitum the fodder and the drinking water. The control and treated chickens were bled gradually in the first day and at every week, respectively. Their blood was collected individually, the blood serum was separated by centrifugation then stored at −18° C. temperature till processing.

The amount of antibodies was determined by ELISA test. In the course of the test the antigen is generally absorbed to the wall of a polystyrene plate with 96 wells. The specific antibodies of the blood serum to be investigated are bound with the antigen, the non-bound antibodies, however, will be removed by washing, then the system will be completed with such a species-specific antiglobulin serum which has been conjugated with horseradish peroxidase enzyme or with another enzyme. The antiglobulin-conjugate molecules failed to enter into the reaction will be removed by washing. The antigen-antibody-antiglobulin conjugate “sandwich” will be made visible in a form of colour reaction by the addition of the enzyme's substrate. In this experiment a kit testing infectious bursitis antibody (ProfFLOK® IBDELISA Kit, manufactured by Kirkegaard & Perry Laboratories, Guilford, UK; catalog number 54-81-01) was employed to the measuring.

The measuring was performed according to the methodology described in the foregoings. To this measuring 50-50 μl serum was added to the wells of the plate sensitized with the antigen. The positive and negative control sera were located in the wells on forepart (−1, +2, −3) and end part (−94, +95, −96) of the ELISA plate. The plates with the serum were incubated for 30 minutes on room temperature, then they were washed with a wash solution (300 μl), the solution remained in the wells for 3 minutes then it was poured down. This washing step was repeated after 2 minutes. Hereupon 100 μl conjugate from the kit was added well by well to the samples then they were incubated for 30 minutes on room temperature, finally they were washed twice according to the foregoings. Hereafter 100 μl substrate was given to the system and it was incubated on room temperature for 15 minutes. The reaction was arrested with 100 μl stop-solution. The developed green-blue color was read in an ELISA reading device at 405-410 nm. The antibody titres were calculated from the obtained absorbance data which were evaluated in weekly breaking-down.

It can be established from these measurings that the titer values of the sera of the animals bled on the 1. day were the same as the normal values of the control group (as an average 13.5). The titer values of the serum samples taken down on the 1. week increased compared to the value of the control group (as an average 17.4). On the 2. week this growth increased further compared to the control (as an average 21.2). On the 3. week a powerful rise in the titer values was observable on the influence of the VET-HBM treatment compared to the control (as an average 30.3). On the 4. week the titer values of the treated groups triplicated compared to the control (as an average 42.1). On the 5. week the titer values were four times higher than the values in the controls (as an average 55.6). On the 6. week the titer values were almost fifth times higher than the values in the control group measured on the 6. week (as an average 69.4).

On the basis of the statistical evaluation it was proved that the values obtained were significant (p<0.001) and they exhibited steep rise compared to the control.

What is claimed is:

1. A method of treating or decreasing the likelihood of an infectious inflammation in an animal comprising:
fermenting wheat germ to obtain a fermented wheat germ extract from the fermented liquid and biomass;
adding the fermented wheat germ extract to a fodder; and
feeding said fodder to an animal, wherein the fermented wheat germ extract is present in said fodder in an amount of 0.1 to 6.0 g/kg fodder, thereby allowing the animal to consume an effective amount of fermented wheat germ extract, also thereby treating or decreasing the likelihood of an infectious inflammation in an animal.

2. A method of treating or decreasing the likelihood of Mycoplasma infection in an animal, which comprises:
fermenting wheat germ to obtain a fermented wheat germ extract from the fermented liquid and biomass;
adding the fermented wheat germ extract to a fodder; and
feeding said fodder to an animal, wherein the fermented wheat germ extract is present in said fodder in an amount of 0.1 to 6.0 g/kg fodder, thereby allowing the animal to consume an effective amount of fermented wheat germ extract, also thereby treating or decreasing the likelihood of Mycoplasma infection.

3. The method according to claim 1, wherein said fermented wheat germ extract is added to a nutriment or premix and said nutriment or premix is fed to the animal.

4. The method according to claim 1, wherein the fermented wheat germ extract is present in the fodder in an amount of 0.001% to 10% by weight.

5. The method according to claim 1, wherein the fermented wheat germ extract and biomass is present in the fodder in an amount of 0.001% to 50% by weight.

6. The method according to claim 1, wherein the fermented wheat germ extract is obtained from a fermented liquid and biomass, and wherein the fermented liquid and biomass is obtained by fermenting wheat germ with Saccharomyces cerevisiae in an aqueous medium.

7. The method according to claim 1, wherein the animal is a farm animal.

8. The method according to claim 1, wherein the Mycoplasma infection is caused by at least one of the Mycoplasms selected from the group consisting of: Mycoplasma gallisepticum, Mycoplasma synoviae, and Mycoplasma hyopneumoniae.

9. The method according to claim 8, wherein the fermented wheat germ extract is present in the fodder in an amount of 0.1 to 6.0 g/kg fodder.

10. The method according to claim 8, wherein the animal is a farm animal selected from the group consisting of: cattle, horse, rabbit, piglet, fattening pig, broiler chicken, egg-laying hen, turkey, goose and duck.

11. The method according to claim 8; wherein the Mycoplasma infection is caused by at least one Mycoplasms selected from the group consisting of: Mycoplasma gallisepticum, Mycoplasma synoviae, and Mycoplasma hyopneumoniae.
